(19) United States
(12) Patent Application Publication
(10) Pub. No.: US 2006/0089667 A1
(43) Pub. Date: Apr. 27, 2006

(54) Apparatus for sealing a puncture in a blood vessel

(76) Inventor: Shlomo Ben-David, TEL-AVIV (IL)

Correspondence Address: FOGG AND ASSOCIATES, LLC, P.O. BOX 581339, MINNEAPOLIS, MN, 55458-1339, US

(21) Appl. No.: 10/515,612

(22) PCT Filed: May 26, 2003
(86) PCT No.: PCT/IL03/00435

§ 371 Date: Aug. 5, 2005

(30) Foreign Application Priority Data
May 27, 2002 (IL) 14958
Apr. 15, 2003 (IL) 155488

(51) Int. Cl. A61B 17/00

(52) U.S. Cl. 606/201

(57) ABSTRACT

An apparatus for sealing a puncture in a blood vessel is disclosed having a tissue-confining device for compressing tissue in the vicinity of the puncture, which longitudinally extends between a proximal end and a distal end and is connected to an adjustable artery clamp for controllably applying pressure, such as by a fluid circuit, onto the blood vessel, so as to reduce the hemostatis time. The artery clamp comprises in a preferred embodiment a proximal plunger and a distal plunger positioned upstream and downstream, respectively, to the puncture. The apparatus is also suitable for treating a pseudoaneurysm.

### FIELD OF THE INVENTION

The present invention is concerned with an apparatus for facilitating sealing of a puncture in a blood vessel during a medical procedure, and a method utilizing the apparatus. More particularly the invention is concerned with an apparatus suitable for cooperation in conjunction with such procedures in which a puncture is formed by a guide sheath introduced into the blood vessel.

### BACKGROUND OF THE INVENTION

During several surgical procedures, for example in treatment of vascular diseases, it is common practice to invade a blood vessel and introduce a treating or diagnostic device, e.g. balloons or various types of stents to operate on walls of the arteries, plaque removing devices, observation and flow diagnostic instruments, etc.

During such procedures, a blood vessel is punctured so as to allow introduction of the instrument through the artery and then maneuver it to the required site of operation. This is carried out in practice by introducing a guide sheath often, through which the instrument can then be easily maneuvered to the site of interest.

A problem occurs once the procedure is complete and the guide sheath has then to be removed, when the percutaneous puncture bleeds. Bleeding may result in hematoma or in severe cases to malfunction of critical organs and even death. Such bleeding is stopped, by a most common method, by simply applying pressure on to the puncture site by a medically trained person for a sufficiently long period of time until hemostasis takes place to spontaneously seal the puncture and stop the bleeding.

In cases of puncturing the femoral arteries, the required time may be as long as about 45 minutes or more and in some cases re-bleeding occurs if the patient is not in rest. Some devices have been proposed for facilitating applying pressure over a blood vessel, some of which particularly for the purpose of sealing a punctured blood vessel. Examples of such devices are disclosed in U.S. Pat. Nos. 3,625,219; 3,884,240; 4,557,262; 5,304,186; and 5,304,201. The devices disclosed in these Patents merely apply mechanical pressure to the blood vessel and do not facilitate use of sealing and flow monitoring devices.

A variety of methods and devices have been suggested for replacing the traditional methods disclosed above, some of which involve introducing chemical compounds which act as hemostatic catalysts or as adhering agents, whilst others aim at introducing various forms of plugging members into the puncture.

The following is a list of prior art patents disclosing devices and methods for sealing punctured blood vessels, all of which being directed to plug-type devices: U.S. Pat. Nos. 4,705,040 4,890,612, 4,929,246, 5,108,420, 5,342,393, 5,350,399, 5,391,183, 5,613,974, 5,810,884, 5,861,003, 5,957,952, 5,984,950, 6,007,563 and WO98/31287.

It is an object of the present invention to provide a novel and inventive apparatus for facilitating effective sealing of a puncture or an incision formed by the introduction of a guide sheath in a blood vessel. A further object of the invention is to provide a method utilizing this apparatus.

It is an additional object of the present invention an apparatus and method for reducing the hemostasis time for a puncture formed by the introduction of a guide sheath in a blood vessel, relative to the prior art.

### SUMMARY OF THE INVENTION

According to a broad aspect of the present invention, an apparatus is provided for entrapping tissue over a punctured blood vessel, in the vicinity of the puncture, prior to withdrawal of the guide sheath, such that effective axial pressure may then be applied at the puncture site, to thereby cause partial or total occlusion of the blood vessel, resulting in that the coagulation process (hemostasis) is more rapid. The apparatus also facilitates easy introduction of sealing means.

According to the present invention, an apparatus for sealing a puncture in a blood vessel comprises a tissue-confining device longitudinally extending between a proximal end and a distal end; at least a proximal plunger positioned adjacent to said proximal end for adjustably applying axial pressure on the blood vessel; said tissue-confining device being connected to an adjustable artery clamp for adjustably applying pressure in an axial direction.

The term artery clamp, as referred to herein, denotes a device and a structure that supports said device, which allows for the compressing of a blood vessel, particularly an artery, by an element-hereinafter referred to as a “plunger”—that is pressed onto said blood vessel. An adjustable artery clamp denotes an artery clamp that may be displaced in a controllable fashion, e.g. wherein the structure is axially and transversally displaceable relative to the puncture site and the pressing elements are axially and longitudinally displaceable relative to the structure.

The term tissue-confining device, as referred to herein, denotes a device with an open area bounded by its frame, which is externally placed over a limb of a patient and above a puncture site of the blood vessel, and is so configured that following the application of an axial force to said tissue-confining device it entraps, within said open area, and compresses tissue in the vicinity of the punctured blood vessel. The depth to which the tissue in the vicinity 6f the punctured blood vessel is compressed depends on the magnitude of said axial force and the rigidity of said tissue. The tissue-confining device is therefore fixated with respect to said blood vessel, and furthermore, reduces the distance between the puncture site and the skin protecting said puncture site. Blood flow through said blood vessel is not necessarily constricted as said tissue in the vicinity of the punctured blood vessel is compressed.

As referred to herein, “axial” means a direction from a plunger to a blood vessel, “longitudinal” means a direction parallel to the axis of a blood vessel and “transversal” means a direction perpendicular to the longitudinal direction. “Proximal” means towards the upstream side of blood flow and “distal” means towards the downstream side of blood flow, relative to a puncture site.

A tissue-confining device, according to an aspect of the invention, comprises two parallel, longitudinally extending bars, interconnected at or adjacent their respective proximal and/or distal ends by arcuate connecting members, said tissue-confining device suitable for compressing tissue in the vicinity of a punctured blood vessel and for being connected to an adjustable artery clamp, which is adapted for adjustably applying pressure in an axial direction onto a blood vessel.

In accordance with a particular embodiment, the apparatus further comprises a distal plunger positioned downstream of said proximal plunger, said distal plunger adapted for applying axial pressure onto the blood vessel, essentially above the puncture site, after withdrawal of the guide sheath.

According to one embodiment, axial pressure is applied by means selected from the group of mechanical means, hydraulic means, pneumatic means and electrical means.

In one aspect, axial pressure is generated by means of a fluid circuit comprising an actuator, a cylinder in which fluid is pressurized, a conduit for said fluid, and a junction by which said cylinder and said conduit are in fluid communication with one another, fluid being flowable within said fluid circuit to or from a plunger.

Preferably, the apparatus further comprises means for adjusting the angular orientation, with respect to the artery clamp, of a point from which pressure is applied to the blood vessel.

According to one embodiment, the tissue-confining device is positioned upstream and adjacent the point of penetration of the guide sheath into the body.

According to another embodiment, the guide sheath is removably attachable to the tissue-confining device.

Noting that the tissue-confining device is fitted with a plunger for applying pressure precisely over the puncture of the blood vessel, it is thus useful in preventing the formation of a pseudoaneurysm, (which is an encapsulated hematoma communicating with an artery, caused by an incomplete sealing of the artery and surrounding tissue after removal of an guide sheath).

Another aspect of the present invention is concerned with an apparatus for controllably applying pressure onto a blood vessel, comprising:

a) at least one axially and longitudinally displaceable plunger for applying pressure onto a blood vessel;

b) means for positioning said at least one plunger to a first location above a skin target and directly over said blood vessel;

c) means for generating a controllable force; and

d) means for transmitting said controllable force to said at least one plunger in such a way that said at least one plunger is axially displaceable from said first location to a second location in contact with said skin target and directly above said blood vessel, and that a controllable and known pressure is applied by said at least one plunger onto said skin target,

said pressure being controllable to such a degree so as to reduce blood flow velocity within said blood vessel.

The means of generating a controllable force is selected from the group of hydraulic means, pneumatic means and electric means.

Another aspect of the present invention is concerned with a method for sealing a puncture in a blood vessel caused by an guide sheath, said method comprising the following steps:
a) confining the blood vessel and fixedly positioning it;
b) applying an axial force on the blood vessel upstream of the puncture, so as to cause partial or total occlusion thereof; and
c) withdrawing the guide sheath.

The term partial occlusion denotes the state at times referred to as stenosis, i.e. where the blood vessel particularly an artery) is only partially occluded, e.g. 50% or more, whereby vibration/pulsation of the artery wall ceases, resulting in the temporary disappearance of the diastole and systole or in reduced blood flow velocity at an arterial puncture site. Partial occlusion reduces the hemostasis time. Indication relating to the extent of occlusion is obtained by measuring blood pressure or blood flow velocity, before and after applying axial pressure to the blood vessel (blood pressure before applying axial pressure may be measured also at the arm of the patient, as known per se), or by measuring the pulses of blood flow within a blood vessel distal to the puncture site.

According to an embodiment of the invention, the method further comprises a step d) wherein axial pressure is applied directly over the puncture.

According to another embodiment, prior to step c) a sealing plug is introduced and placed over the puncture. In one aspect, the sealing plug is slid over the guide sheath, is downwardly displaced, and introduced into the blood vessel at the puncture site.

A sealing plug used in conjunction with the present invention comprises a blood vessel engaging portion for bearing against the boundaries of the puncture, and a sealing portion slidably received over the guide sheath; said sealing portion being spontaneously sealable upon withdrawal of the guide sheath; the sealing plug being displaceable by a pusher member.

In one aspect, a sealing plug connected to the bottom of a telescoping plunger is slidable about the outer wall of the guide sheath. The telescoping plunger is retracted or extended by means selected from the group of mechanical means, hydraulic means, pneumatic means and electrical means.

Another aspect of the present invention is concerned with a method for sealing a puncture in a blood vessel caused by a guide sheath, said method comprising the following steps:
a) axially positioning over the blood vessel, at the vicinity of the puncture, an apparatus comprising a tissue-confining device and a proximal plunger positioned upstream of the puncture, said tissue-confining device connected to an adjustable artery clamp device;
b) adjustably applying an axial force at the vicinity of the puncture by said artery clamp device, to thereby confine the blood vessel;
c) adjustably applying axial pressure on the blood vessel by said proximal plunger, to reduce the blood pressure and blood flow in the blood vessel;
d) withdrawing the guide sheath.

Another aspect of the present invention is concerned with a method for treating a pseudoaneurysm, said method comprising the following steps:

a) detecting a pseudoaneurysm and a pseudoaneurysm neck between an artery and said pseudoaneurysm;

b) confining said pseudoaneurysm or pseudoaneurysm neck;

c) fixedly positioning said pseudoaneurysm or pseudoaneurysm neck;

d) applying a controllable axial force on said artery upstream to a puncture which resulted in said pseudoaneurysm, so as to cause partial or total occlusion within said artery; and

e) applying a controllable axial force on said pseudoaneurysm neck, thereby inducing a blood clot within said pseudoaneurysm.

### BRIEF DESCRIPTION OF THE DRAWINGS

In the drawings:

FIG. 1 is a front isometric view of an apparatus according to an embodiment of the present invention;

FIG. 2 is a detailed isometric view of a tissue-confining device according to an embodiment of the invention, also showing a portion of the artery clamp device;

FIG. 3 is a side view of the apparatus illustrated in FIG. 1, in use during a medical procedure, over a patient's limb;

FIG. 4 illustrates the device of FIG. 2 used in conjunction with a transducer of an imaging device;

FIGS. 5A to 5G illustrate variations of a tissue-confining device in accordance with embodiments of the present invention;

FIGS. 6A and 6B are an isometric and a side view, respectively, of a tissue-confining device according to still an embodiment of the invention, also showing a portion of the artery clamp device, in two respective positions;

FIG. 7A is an isometric view of a puncture sealing plug for use in conjunction with the present invention;

FIG. 7B illustrates the sealing plug of FIG. 7A fitted with an introducer therefore;

FIG. 8 is a front isometric view of a hydraulically powered artery clamp device, according to another embodiment of the invention;

FIG. 9 is a rear isometric view of the embodiment of FIG. 8;

FIGS. 10A-C are isometric views of three configurations of a tissue-confining device, respectively;

FIG. 11A is a vertical cross sectional view of a portion of a hydraulic circuit according to one embodiment of the invention, showing an actuator and a junction;

FIG. 11B is a perspective view of the actuator of FIG. 11A;

FIG. 12 is a front isometric view of an artery clamp device according to the invention, showing the position of a tissue-confining device relative to an arterial puncture site;

FIGS. 13A and 13B are a side and a vertical cross sectional view of a plunger casing, showing one variation of a pressure pad;

FIG. 14 is a front isometric view of an artery clamp device according to the invention, showing another variation of a pressure pad;

FIGS. 15A and 15B are side isometric views of a retracted and extended telescoping plunger, respectively, by which a sliding plug is displaced along an guide sheath;

FIG. 16 is a front isometric view of a hydraulically displaceable sealing plug; and

FIG. 17 is a front isometric view of a mechanically displaceable sealing plug.

### DETAILED DESCRIPTION OF PREFERRED EMBODIMENTS

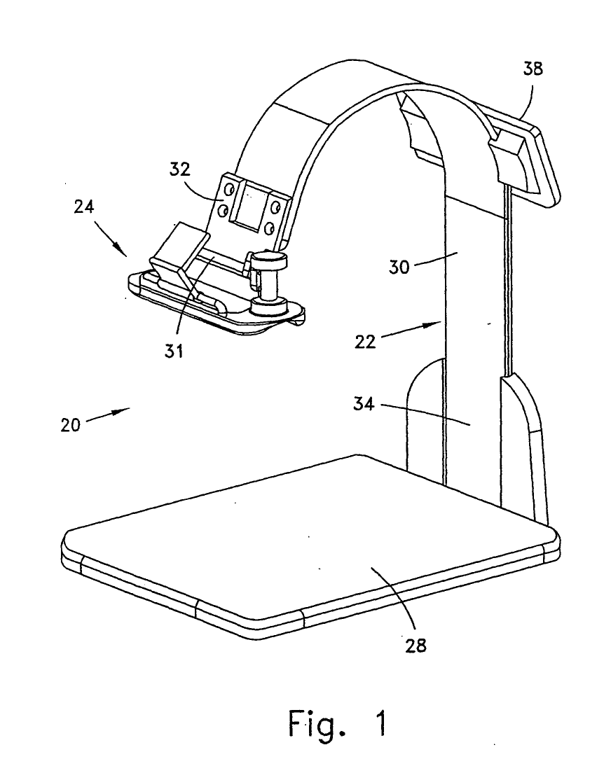

Attention is first direct to FIG. 1 which is an overall view of an apparatus in accordance with the present invention generally designated 20, comprising an adjustable artery clamp device designated 22 and a tissue-confining device generally designated 24.

Artery clamp device 22 comprises a base plate 28 from which vertically extends an adjustable arm 30 fitted at a free end thereof with an attachment bracket 32.

Arm 30 may be adjusted as far as the height of adjustment bracket 32 from the base plate 28 and the transversal distance of the adjustable bracket 32 from the essentially vertical leg portion of the arm 30.

An artery clamp device in accordance with the invention may be any suitable such device in which its free end may be displaced both vertically and horizontally to correspond with different sizes and locations over a patient's limb and for applying suitable axial pressure. This may be obtained by different mechanical, hydraulic, pneumatic or electrical means as known per se.

A pressure indicator 38 is mounted on the arm 30 for indicating the pressure applied at the free end thereof Such a pressure indicator may be associated with a strain gauge or pneumatic means, as known per se.

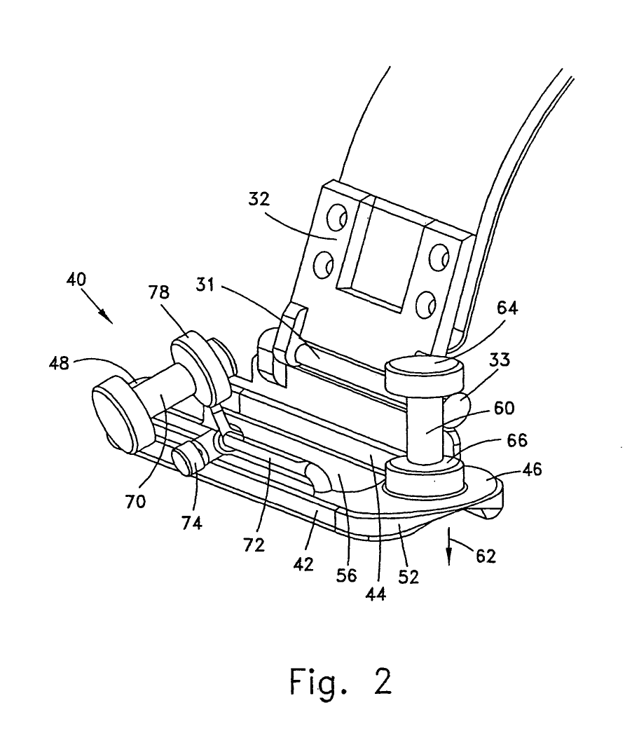

Tissue-confining device 24 is pivotally attached to the adjustment bracket 32 of the artery clamp device at 31 and the angular position of the tissue-confining device may be changed with respect to the artery clamp device by release knob 33 (see also FIG. 2). The angular orientation of the tissue-confining device with respect to the artery clamp is fixable.

Further attention is now directed to FIG. 2 illustrating in more detail a particular embodiment of a tissue-confining device generally designated 40. The device in the present embodiment is a frame-like member comprising two longitudinally extending bars 42 and 44, the length of which may range from 20-90 mm, connected to one another at a proximal end 46 and a distal end 48. The term proximal end corresponds with the position of the device during the course of operation, with an upstream side of a blood vessel at the patient's limb.

As noted, the bars 42 and 44 are connected to one another by a respective proximal connecting bar 52 and a distal connecting bar (not seen in FIG. 2), both being arcuate in a concave manner. Longitudinally extending bars 42 and 44 and the connecting bars define an open area 56 formed therebetween.

Proximal plunger 60 fitted over the proximal connecting bar 52 is adapted for applying axial pressure in the direction of arrow 62. The plunger 60 may be temporarily depressed by applying pressure over tab 64 or may be fixed at any axial extent by retention nut 66. The purpose of plunger 60 will become apparent hereinafter.

A distal plunger 70 is slidably and pivotally mounted on a longitudinally extending rod 72 and its angular and longitudinal positions may be securely fixed by means of a spring-biased fixation knob 74. Plunger 70, similarly to plunger 60, may be temporarily depressed or may be fixedly retained at any axial extent by means of retention nut 78. The purpose of this plunger will also become apparent hereinafter.

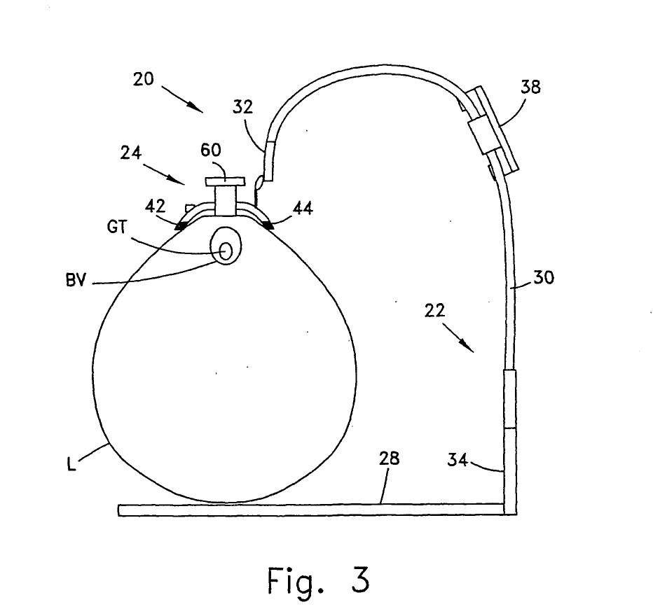

Further attention is now directed to FIG. 3 illustrating how an apparatus 20 in accordance with the present invention is in actual use. The base plate 28 of the artery clamp device 22 is placed under the patient's limb L (typically a thigh), for which the medical procedure is to be carried out. The arm 30 of the artery clamp device is then adjusted such that the tissue-confining device 24 tightly bears over the limb L at the site of puncture, and applies axial pressure thereto, such that a portion of tissue becomes confined between the bars 42 and 44 of the tissue-confining device and deforms to project into the open area 56 (FIG. 2). Tissue-confining device 24 is therefore positioned and fixated in relation to blood vessel BV, such that the latter longitudinally extends through open area 56, passing directly underneath plunger 60. In FIG. 3, a guide sheath GT is received within the blood vessel BV. The pressure applied onto the limb L by plunger 60 is monitored by the pressure indicator 38.

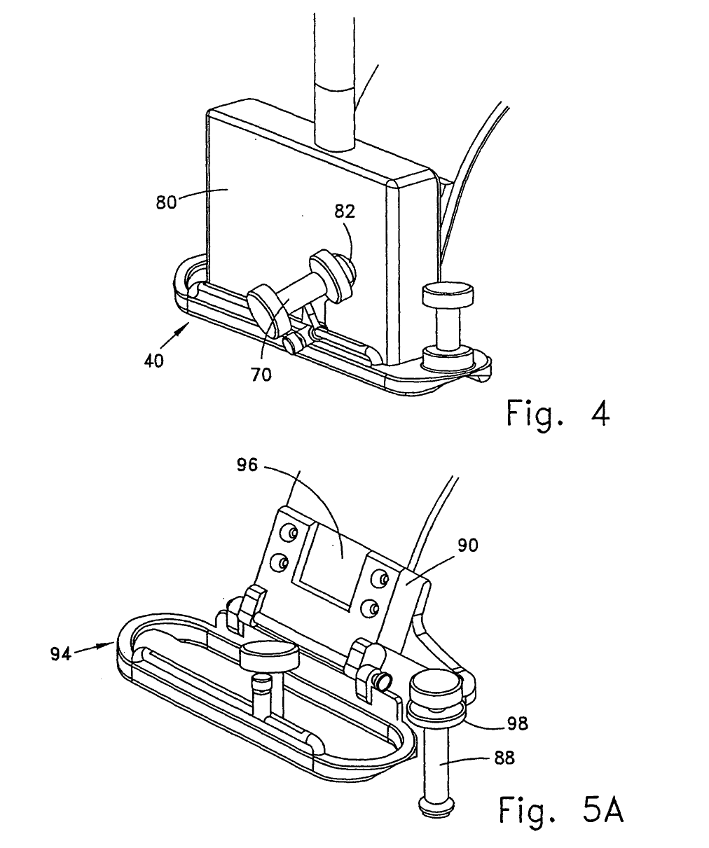

The embodiment of FIG. 4 illustrates a tissue-confining device 40, identical with that illustrated in detail in FIG. 2, while distal plunger 70 is shifted away from the open area 56 so as to clear the space, allowing for the accommodation of a transducer 80 of an imaging device in a fixed manner. The imaging device may be an ultrasound device, such as an X-ray imaging device, for providing required data concerning blood flow through the artery and to provide indication corresponding with the precise position of the puncture in the blood vessel. The transducer 80 may be fixedly nested within a seating of the tissue-confining device, e.g. by means of plunger 70 engaging with a suitable recess 82 formed on the transducer 80 which facilitates attachment or detachment of plunger 70.

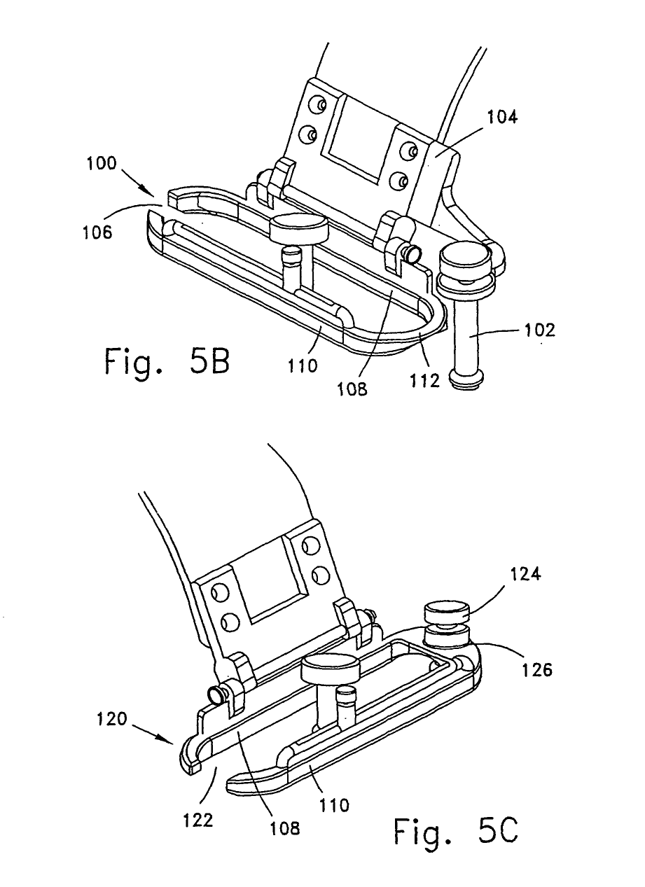

FIGS. 5A to 5G illustrate modifications of tissue-confining devices according to the present invention. In FIG. 5A proximal plunger 88 integrally extends from the attachment bracket 90, (attaching the tissue-confining device 94 to the free end 96 of the artery clamp device-not shown). Here again, the axial displacement of the proximal plunger 88 may be adjusted and temporarily fixed by means of retention nut 98. Other elements of the tissue-confining device 94 are similar with those disclosed in connection with the embodiment of FIG. 2.

The tissue-confining device 100 illustrated in FIG. 5B is principally similar to that disclosed in FIG. 5A wherein the proximal plunger 102 integrally extends from bracket 104 rather than being integral with the tissue-confining device. A difference however resides in that the longitudinally extending bars 108 and 110 of the tissue-confining device are connected to one another only by a proximal connecting bar 112, leaving an opening 116 at the distal end, for receiving therethrough a guide sheath (as exemplified in FIG. 5D).

Similarly, the embodiment of FIG. 5C illustrates a tissue-confining device 120 formed with a distal opening 122. However, in this embodiment the proximal plunger 124 is integral with the proximal connecting bar 126 rather than with the bracket connecting to the free end of the artery clamp device as in FIG. 5B.

In FIG. 5D, the tissue-confining device 132 has a distal opening 134, as in FIGS. 5B and 5C, adapted for placing the tissue-confining device 132 over a guide sheath 136 such that the guide sheath extends within the open area 138 of the tissue-confining device. This allows for positioning of the distal plunger 139, which is a rectangular element, directly above the puncture in the blood vessel.

The distal plunger 139 of FIG. 5D functions similarly to that disclosed in connection with FIG. 2, with the difference that it cannot be fixed at different axial positions but rather is pressured by the finger tips of an operator to the required degree of pressure.

FIGS. 5D and 5F illustrate embodiments of tissue-confining devices in which the guide sheath extends within the gap, whilst FIG. 5E illustrates a tissue-confining device in which the guide sheath extends behind (i.e. downstream) the distal connecting bar of the tissue-confining device.

In order to increase the contact area of the tissue-confining device with the tissue in the vicinity of the blood vessel, the proximal end of tissue-confining device 146 in FIG. 5E comprises two proximal extensions 148 and 150, which result in an open area 152 formed therebetween.

FIGS. 5F and 5G illustrate a modification of a tissue-confining device generally designated 160 in which a front bar 162 is formed with an opening 164 of width corresponding with the width of a guide sheath 168, or greater.

The proximal plunger 170 is integral with the proximal connecting bar 172. The distal plunger 176 is slidably received on a rod 178 which in turn is pivotally mounted at 182 to the front bar 162, whereby it is pivotal between an open position (FIG. 5F) and a closed, operative position (FIG. 5G).

The open position of FIG. 5F is useful for removing the guide sheath 168 prior to withdrawal thereof from the limb (not shown). After withdrawal of the guide sheath 168 the distal plunger 176 may be used for applying direct axial pressure over, the puncture site.

The procedure in accordance with the present invention is such that after completing the medical procedure performed by a stent (introduced through the guide sheath), the adjustable artery clamp device 22 (FIG. 3) is placed such that the base plate 28 bears under the limb L, and arm 30 is then adjusted so that the distal and proximal plungers may apply direct axial pressure above the blood vessel BV (an artery in the present case) through which the guide sheath GT extends. The axial pressure applied by a plunger, or alternatively by a tissue-confining device, is monitored by pressure monitor 38. The arrangement is such that a tissue portion is compressed by the side bars 42 and 44 of the tissue-confining device, and adjoining tissue projects into the open area between the longitudinally extending bars, as illustrated in FIG. 3. As a result, the tissue-confining device is positioned and fixated relative to the blood vessel.

Then, the proximal plunger 60, extending upstream of the puncture formed by the guide sheath GT, is lowered so as to cause partial occlusion known as stenosis) at the rate of 50% or more. This situation eliminates the vibrating/pulsating effect of the walls of the artery, resulting in a lack of systole and diastole, as well as in reduced blood flow velocity at the puncture site. It is well known that in the absence of such vibrations/pulsations improved coagulation occurs, as the blood platelets accumulate easier at the puncture of the blood vessel, reducing the coagulation (hemostasis) time. The extent to which the blood vessel is occluded can be monitored by a suitable transducer as illustrated in FIG. 4. This may be carried out by measuring the blood flow pressure or velocity, or by measuring the pulses of blood flow within a blood vessel distal to the puncture site.

At the next stage, the guide sheath GT may be withdrawn, with the proximal plunger still applying axial pressure upstream, maintaining low blood flow and pressure downstream from the puncture site.

In accordance with another embodiment, where the tissue-confining device comprises also a distal plunger (as in the exemplified embodiments hereinabove), upon withdrawal of the guide sheath GT, the distal plunger (not seen in FIG. 3) is axially lowered directly above the puncture at the blood vessel, improving and increasing speed of sealing of the puncture by coagulation with respect to prior art puncture sealing devices. Such pressure may be applied for as long as may be required from several minutes to as much as an hour or so. At any point of time, the transducer may be placed at the tissue-confining device for obtaining information regarding blood flow within the blood vessel at the puncture site.

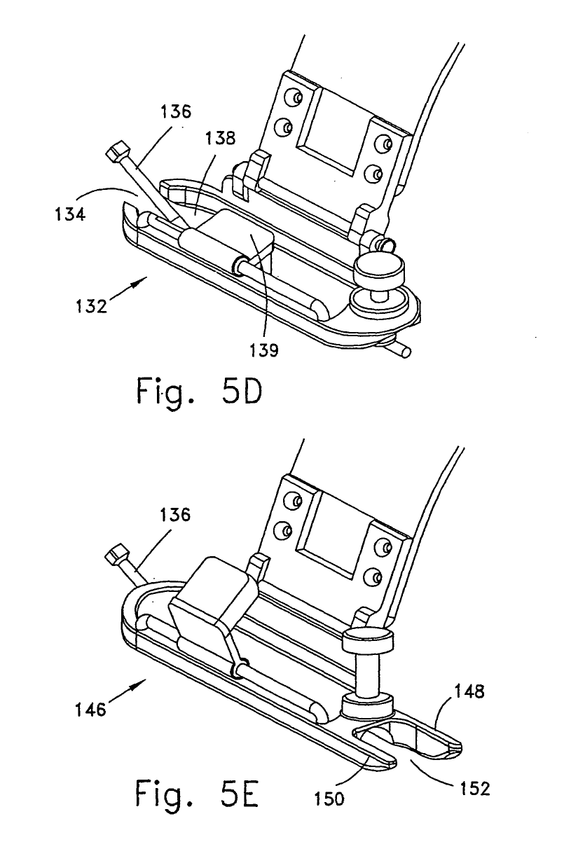

In FIGS. 6A and 6B, an additional embodiment of a tissue-confining device in accordance with the invention is illustrated, generally designated 190, which differs from the previous embodiments in that the proximal plunger is fitted on a bar 198 which is slidingly received, by a dovetail arrangement, to a corresponding groove 200 of the bracket 202, whereby the proximal plunger is longitudinally displaceable with respect to the tissue-confining device 190, to allow for adjusting the location of the pressure point being applied thereby on the blood vessel (not shown). Similarly, a distal plunger 206 is provided, which is mounted on a bar 208 slidingly received, by a dovetail arrangement, to a corresponding groove 210 of the bracket 202, such that the distal plunger 206 is longitudinally displaceable with respect to the tissue-confining device 190, to allow for adjusting the location of the pressure point being applied thereby on the blood vessel (not shown).

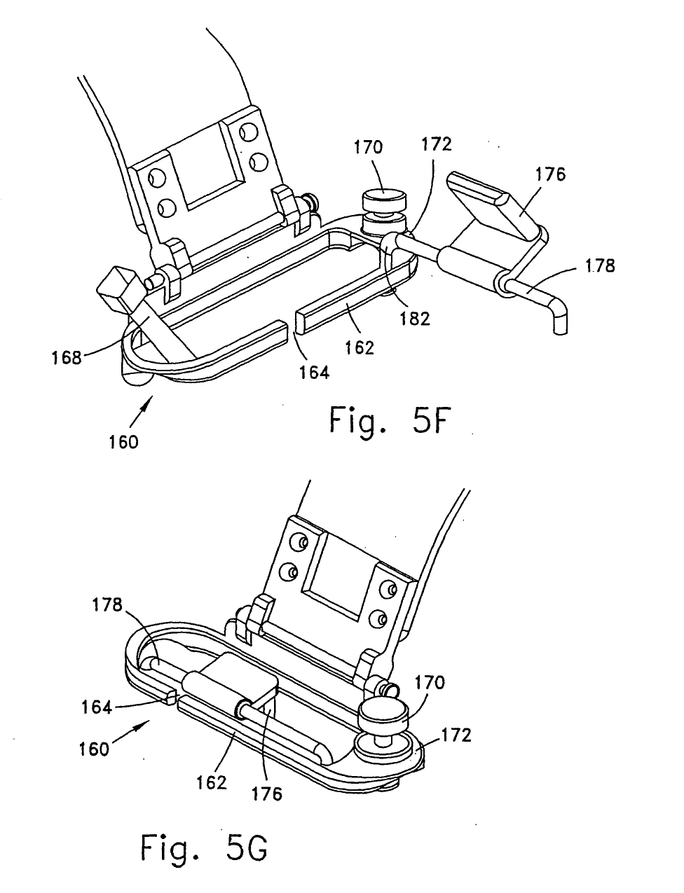

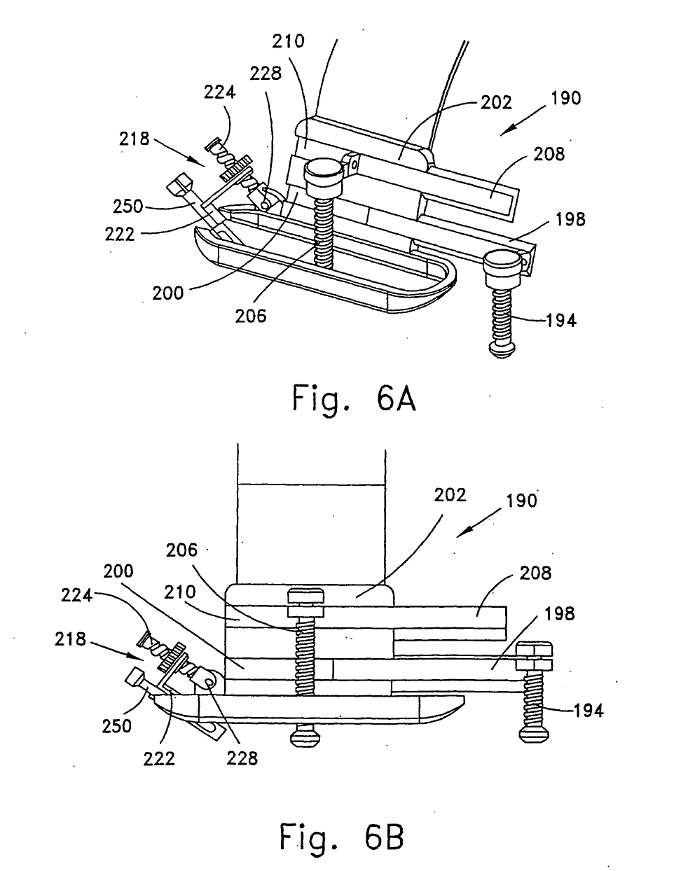

Further illustrated in FIGS. 6A and 6B is a sealing plug support device 218 adapted for positioning and supporting a sealing plug pusher member 250 (illustrated in more detail in FIG. 7B), comprising a support sleeve 222 mounted on a screw-type rod 224, which in turn is pivotally connected to the tissue-confining device 190 at 228. The sealing plug support device 218 facilitates supporting the pusher member 250 at a desired angular position and for axial displacement thereof by means of the threaded rod 224. The pusher member may be integral with the sealing plug.

In accordance with still an embodiment of the invention, a sealing plug 240 (FIGS. 7A and 7B) may be used in conjunction with the apparatus according to the invention. Sealing plug 240 is a tubular body formed at a top end thereof with a sealing portion which is a pre-slotted resilient membrane 244, and having a truncated bottom wall 246, corresponding with a typical angle of insertion of the guide sheath (not shown). The body and the pre-slotted resilient membrane are sized so as to facilitate sliding thereof over an guide sheath. A pusher member 250 (FIG. 7B) is attached to the sealing plug 240 sized for sliding over the guide tube GT (guide sheath) so as to facilitate withdrawal of the guide sheath therethrough.

Prior to withdrawal of the guide tube GT, the punctured sealing plug 240 (FIG. 7A) is slidingly displaced along the guide sheath by means of the dispensing and guide tube 250 until a bottom surface 246 of the sealing plug 240 engages the blood vessel at the site of the puncture, such that upon withdrawal of the guide sheath through the sealing plug, the pre-slotted resilient membrane spontaneously seals and the surface 246 applies direct pressure over the puncture. After a while, the sealing plug 240 is removed by the aid of a dispensing tube 250 and then, further axial pressure may be applied by the distal plunger as discussed hereinabove. The hemostatis time may be further reduced by applying a hemostatic sealant, e.g. biodegradable materials, such as collagen, gelatin, fibrinogene, oxidized cellulose, hyaluronic acid, and crosslinked dextrans, onto the sealing plug. Alternatively, the sealing plug may be made from a biodegradable material, and therefore may remain at the arterial puncture site while continuing to apply pressure thereat.

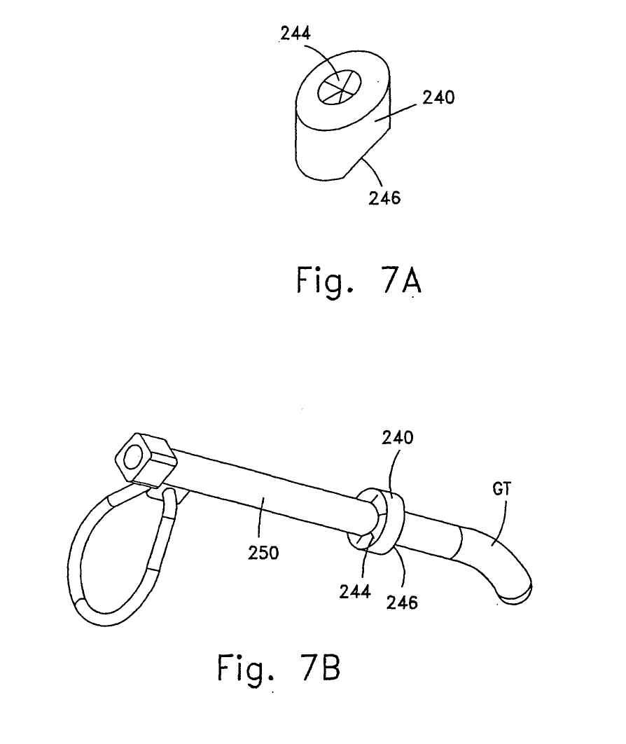

FIGS. 8-14 illustrate another embodiment of the invention wherein the distal and proximal plungers apply pressure on a blood vessel by hydraulic means, in order to further reduce the hemostasis time, in accordance with the present invention. Although the following description relates to a hydraulic means for applying pressure onto a blood vessel, it will be appreciated that the apparatus can be similarly employed for applying pressure by pneumatic means, and any reference hereinafter to “hydraulic fluid” is also applicable to air, or any other compressible fluid.

As shown in FIG. 8, the artery clamp device, designated generally as 300, comprises axially displaceable distal plunger 330, proximal plunger 360, and an angularly displaceable pad 310 connected to each of the distal and proximal plungers. A hydraulic circuit comprising mechanical actuator 285, cylinder 290 in which hydraulic fluid is pressurized, e.g. ranging from 10 to 40 psi, conduit 325 for the hydraulic fluid, manometer 350 for indicating the level of applied pressure on the tissue and junction 335, by which cylinder 290, conduit 325 and manometer 350 are in fluid communication with one another, are provided with each of the distal plunger 330 and proximal plunger 360.

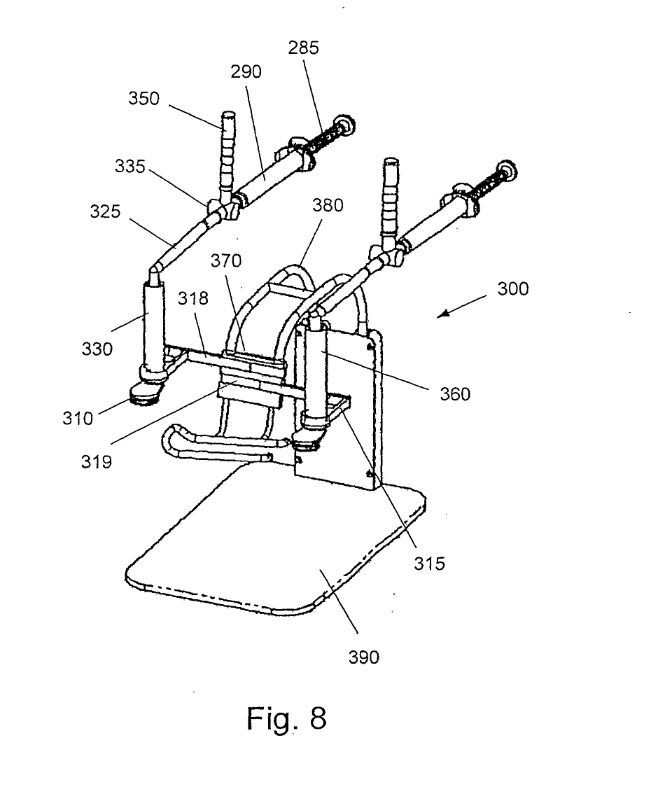

Adapter 370 facilitates positioning of plungers 330 and 360. Distal plunger 330 and proximal plunger 360 are longitudinally displaceable by means of a corresponding slider 318 of rectangular cross section, which is slidingly received, by a dovetail arrangement, within a corresponding groove 319 formed within adapter 370. A corresponding leg 315 connects each of the plungers 330 and 360 to a corresponding slider 318. Axial positioning of adapter 370, and consequently of plungers 330 and 360 prior to being repositioned by means of hydraulic fluid, relative to base plate 390 is effected by lowering or raising arcuate arms 380 received in the top of adapter 370. After displacing adapter 370 to a desired axial position, lock screw 387 secures legs 385 of corresponding arcuate arms 380 to socket 386, which is perpendicular to base plate 390, as shown in FIG. 9.

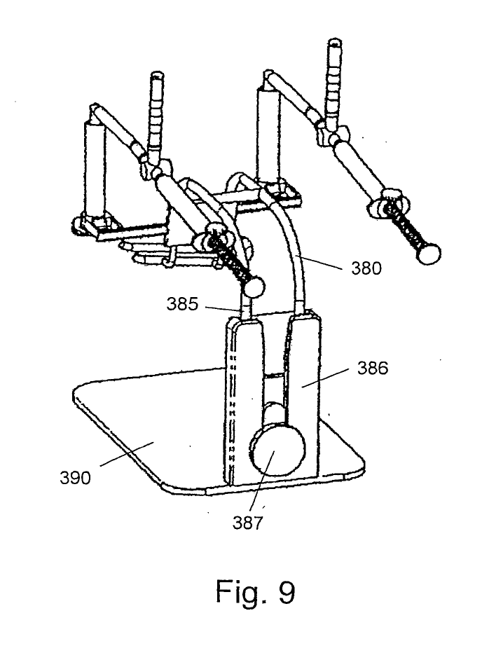

Adapter 370 is also adapted to receive a tissue-confining device. Various configurations of tissue-confining devices may be employed, such as devices 410A-C, as illustrated in FIGS. 10A-C, respectively. Each tissue-confining device is provided with a pair of connectors 420, which are integrally formed therewith in such a way, e.g. with a 90-degree elbow, that each connector is spaced from the longitudinally extending bar 425 of the tissue-confining device positioned adjacent to the adapter, and is therefore engageable with the adapter, e.g. by a pressure fit, at the bottom thereof, without interfering with the axial pressure applied by the longitudinally extending bars. Accordingly, the axial position of adapter 370 relative to base plate 390 during a puncture sealing procedure is preferably such that a selected tissue-confining device will apply axial pressure to the underlying tissue.

Referring now to FIGS. 10A-C, the distance between the two longitudinally extending bars 425 of each tissue-confining device, e.g. ranging from 5-40 mm, is selected so that the transversal spacing between an artery, within which a catheter was guided during a recent surgical procedure, and each bar 425 ranges from 1-3 cm. With such a transversal spacing, the two bars 425, which are immobilized while being in pressing engagement with tissue at the point of resistance of said tissue and may be supported by a bone in the vicinity of the artery, when a tissue-confining device applies axial pressure to the underlying tissue, the artery is fixated by compressed tissue that is interposed between the artery and each bar 425. Base plate 390 (FIG. 8) placed underneath a patient further supports and stabilizes the tissue-confining device.

In the shown configuration of a tissue-confining device, each connecting bar 427 which connects the two longitudinally extending bars 425, whether at the distal or proximal end thereof, is provided with a curvature with respect to a vertical plane, such that the connecting bar is axially separated from a longitudinally extending bar. This curvature retains mechanical integrity of the tissue-confining device without compressing the artery, since the connecting bar is not in contact with the tissue. In FIG. 10A, two connecting bars 427 are employed, while in FIG. 10B only one is used, with an opening 428 being formed at the proximal end of tissue-confining device 410B. Opening 428 advantageously allows for the placement and repositioning of an imaging device. In FIG. 10C, the distal end of tissue-confining device 410C is provided with two ends 429, which are in a spaced, opposed relation with one another, having a curvature with respect to a vertical plane.

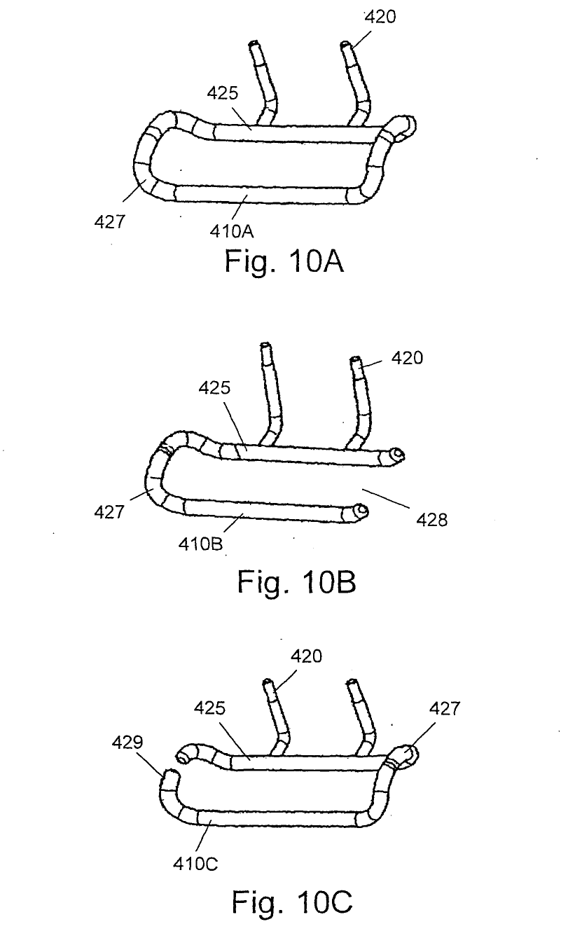

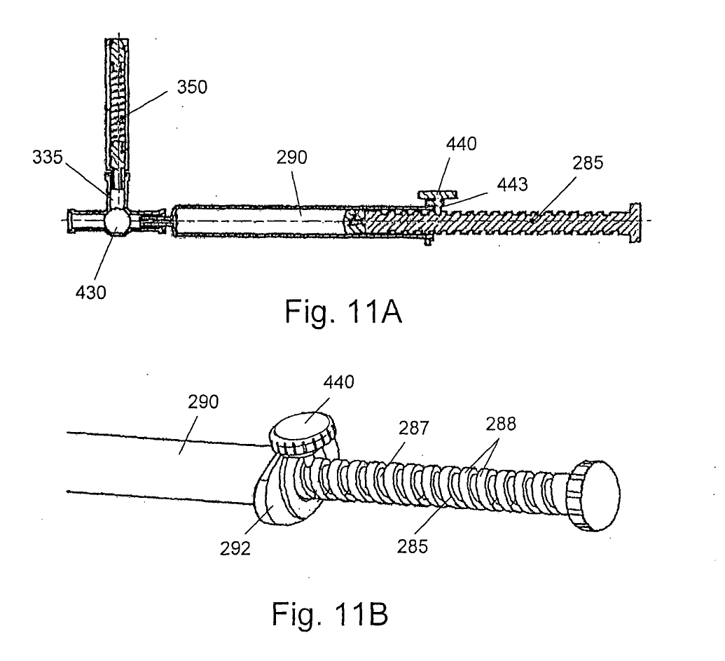

After inserting a desired tissue-confining device and axially and longitudinally positioning plungers 330 and 360, pressurized hydraulic fluid may be delivered to the plungers, for the lowering of the latter on selected locations along an artery, e.g. the femoral artery. Hydraulic fluid is introduced to cylinder 290 via an opened stopcock (not shown), or via any other suitable valve in communication with port 430 of junction 335 (FIG. 11A), and is pressurized within the cylinder, after the valve is closed, by advancing actuator 285 in the direction of manometer 350. As the pressure of hydraulic fluid rises within cylinder 290, fluid is delivered to the corresponding plunger, after flowing through conduit 325, due to the pressure differential that initially exists between the cylinder and the corresponding plunger, whereupon the corresponding plunger contacts the underlying tissue. Due to the reactive force applied by the tissue onto the plunger, the pressure of the hydraulic fluid rapidly increases to a level ranging from 10 to 40 psi. The pressure applied by a plunger onto the tissue, which is indicated by manometer 350, is controllable, as will be described hereinafter. Upon achieving a desired applied pressure, lock screw 440 secures actuator 285 to a fixed position (FIGS. 11A-B). The hydraulic fluid is self-contained in a closed hydraulic path between a plunger and a corresponding cylinder 290, and therefore may be drawn from a plunger to the corresponding cylinder 290 upon conclusion of the puncture sealing procedure by retracting actuator 285 and reducing the pressure of the hydraulic fluid by increasing the volume of cylinder 290.

As shown in FIGS. 11A and 11B, actuator 285 is cylindrical and is formed with threading. The external threading of the actuator facilitates the securing of lock screw 440, which protrudes from lip 292 of cylinder 290, to actuator 285, i.e. as a result of the pressing of pin 443 of the lock screw to a flank 287 located between adjacent threads 288. If the actuator were not secured to the cylinder, the actuator would be rearwardly displaced and consequently released from the cylinder, due to an increase in pressure within the corresponding plunger. In addition to providing a securing means, the external threading also provides a means of displacing the actuator within cylinder 290, in order to pressurize the hydraulic fluid. By securing lock screw 440 at a moderate pressure to actuator 285, the actuator is prevented from being rearwardly displaced, yet the actuator is not immobilized and may be rotated within cylinder 290. As actuator 285 is rotated, pin 443 of the lock screw continuously contacts the flank 287 between a changing pair of adjacent threads 288. If so desired, pin 443 may be lowered below the height of threads 288 without being in contact with flank 287. Rearward movement of the actuator is prevented due to the contact of pin 443 with a thread 288, while its rotation is made possible through a path between adjacent threads. Consequently, the pressure applied by a plunger is controllable by gradually advancing or retracting the actuator when the plunger is in contact with the underlying tissue, in response to the pressure indicated by the manometer. For example, the pressure applied by the proximal cylinder is generally increased until a reduction in blood flow velocity is noticeable or partial occlusion within the blood vessel is achieved.

By employing a three-way or four-way stopcock, the pressurized hydraulic fluid may be isolated from junction 335 after having been delivered to a plunger. It will therefore be appreciated that one actuator 285 and one cylinder 290 may be used for both the distal and proximal plungers. That is, the actuator and cylinder may be removed from a junction 335 after hydraulic fluid has been delivered to the proximal plunger, for example, and isolated from its corresponding junction, and then the same actuator and cylinder may be used for delivering hydraulic fluid to the distal plunger.

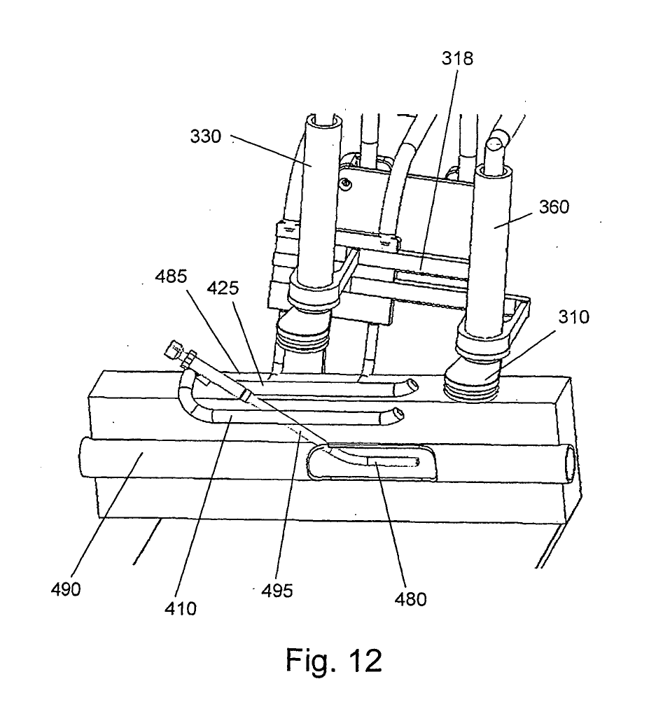

FIG. 12 illustrates a typical positioning of tissue-confining device 410 relative to a puncture site 480 of the femoral artery. As previously mentioned, precise positioning of a tissue-confining device is carried out in conjunction with the data transmitted by a duplex ultrasound, Doppler, or any other imaging device, in order to determine the exact anatomical location of the femoral artery and the arterial puncture site, as well as the blood flow velocity, so that an optimal magnitude of axial pressure may be applied to the tissue. After determining the exact location of the artery and arterial puncture site, tissue-confining device 410 is transversally positioned such that the axis of the femoral artery 490 is parallel to, interposed between, and located below, the two longitudinally extending bars 425 of the tissue-confining device. Furthermore, tissue-confining device 410 is longitudinally positioned so that arterial puncture site 480 is located at substantially the transversal centerline of bars 425. Pressure applied to the underlying tissue 485 by a correctly positioned tissue-confining device results in fixation of the two bars 425 relative to artery 490 and reduces the tissue thickness between the skin and the artery. Consequently the pressure applied by distal plunger 330 and proximal plunger 360 to the artery may be effected more rapidly and accurately than with prior art puncture sealing devices, resulting in a significant reduction of hemostatic time. Furthermore, pressure applied to the tissue by a tissue-confining device decreases the angle of a wound canal, which is formed by the penetration of guide sheath 495 into tissue 485 for the purpose of cardiac catheterization, with respect to a horizontal plane, thereby facilitating the sealing of the wound canal.

Proximal plunger 360 is adapted to apply a sufficient axial pressure to artery 490, at a location of 1 to 5 cm proximal to arterial puncture site 480, in order to induce moderate stenosis, severe stenosis or total occlusion within the artery. It will be appreciated that minimal blood flow through artery 490 is retained so as to prevent premature clot disintegration, and consequently to reduce risks of bleeding, pseudoaneurysm and hematoma. The partial retraction of guide sheath 495, concurrently with the lowering of proximal plunger 360, initiates blood flow through the wound canal. A sufficient interruption of femoral arterial flow may be ascertained by imaging means, or alternatively, by measuring pedal or politeal pulses, at a location distal to arterial puncture site 480, or by visually determining lack of blood seepage from the wound canal. The proximal plunger is preferably lowered onto the selected pressure point in a single continuous motion, so as to minimize patient discomfort.

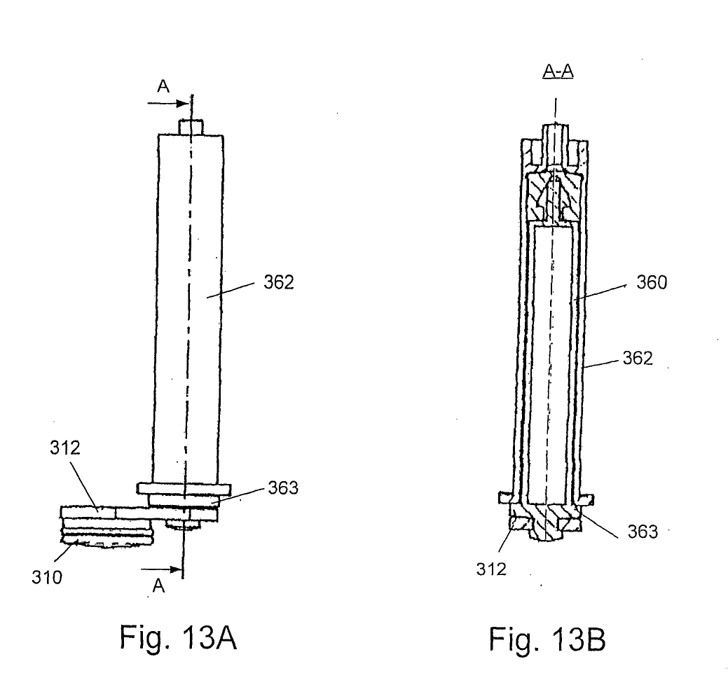

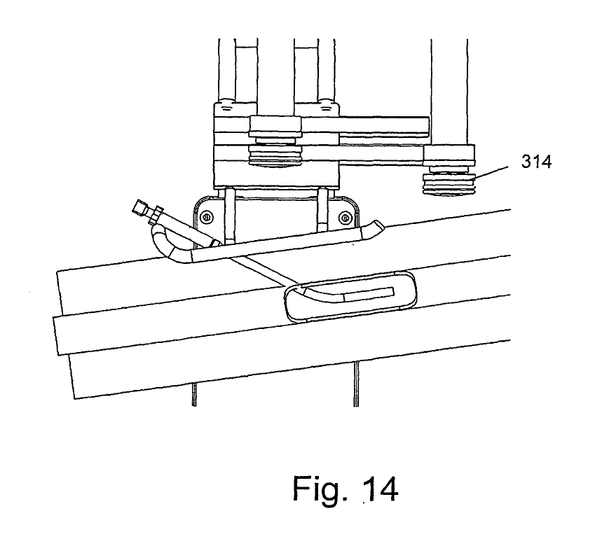

If pad 310 of the proximal plunger is not directly located above artery 490 at such a distance from arterial puncture site 480, following longitudinal displacement of slider 318, it may be rotated to ensure axial compression directly on the correct location of the artery. As shown more clearly in FIGS. 13A and 13B, plunger 360 is rotatable within its casing 362. Since elliptical upper surface 312 of pad 310 is connected to flange 363 of the plunger by a press fit as shown, or alternatively may be integrally formed therewith, pad 310 is also rotatable about the axis of plunger 360. Therefore the pad serves as an angular adjusting means. If so desired, the proximal plunger may terminate with a concentric, circular pad 314 as shown in FIG. 14. Distal plunger 360 is provided with a similar pad arrangement.

Distal plunger 330 is adapted to apply axial pressure directly over arterial puncture site 480. Since the distal plunger may be advantageously longitudinally and axially positioned in a speedy manner, a physician performing the puncture sealing procedure can apply pressure to arterial puncture site 480 within 3 minutes, for example. The pressure of the hydraulic fluid delivered to the distal plunger may be lower than that delivered to the proximal plunger, a level ranging e.g. from 5 to 20 psi. After the distal plunger begins to apply pressure, the pressure applied by the proximal plunger may be gradually decreased in small increments, in order to prevent premature clot disintegration. By applying a compressive force at two pressure points, rather than at one pressure point, the pressure applied by each of the proximal and distal plungers is therefore reduced.

Although the aforementioned description related to the coagulation of a puncture site produced within an artery, and particularly the femoral artery, it will be appreciated that the use of the present invention is suitable for any blood vessel. Accordingly, the present invention is also applicable for the treatment of a pseudoaneurysm and the absorption thereof into an adjacent blood vessel, by the application of an axial force by the proximal plunger proximally to a puncture site and by the distal plunger on the path of blood communication between the artery and the hematoma (commonly referred to as the pseudoaneurysm neck). For a pseudoaneurysm neck having a length less than 5 mm, the pressure applied by the distal plunger thereon usually suffices to prevent blood inflow into the pseudoaneurysm. However, if the length of the pseudoaneurysm neck is greater than 5 mm, supplementary axial pressure applied to the pseudoaneurysm neck is provided by a longitudinally extending bar of the tissue-confining device. A clot may therefore be formed in the pseudoaneurysm in approximately 15 minutes, in contrast with a duration of approximately 1.5 hours that is needed with prior art pseudoaneurysm treatment methods whereby pressure is applied directly onto the puncture site.

It will be appreciated that the distal and proximal plungers may be similarly electrically actuated, e.g. while in communication with a controller, as is well known to those skilled in the art, in accordance with the aforementioned puncture sealing method

Another embodiment of the invention incorporates a hydraulically displaceable sealing plug for augmenting the aforementioned puncture sealing method, particularly suitable for punctures caused by large sheaths of greater than 8 French (an inner diameter of greater than 2.64 mm). Once again, the following description relates to hydraulic means, but it will be appreciated that pneumatic and electric means may also be employed to displace the sealing plug.

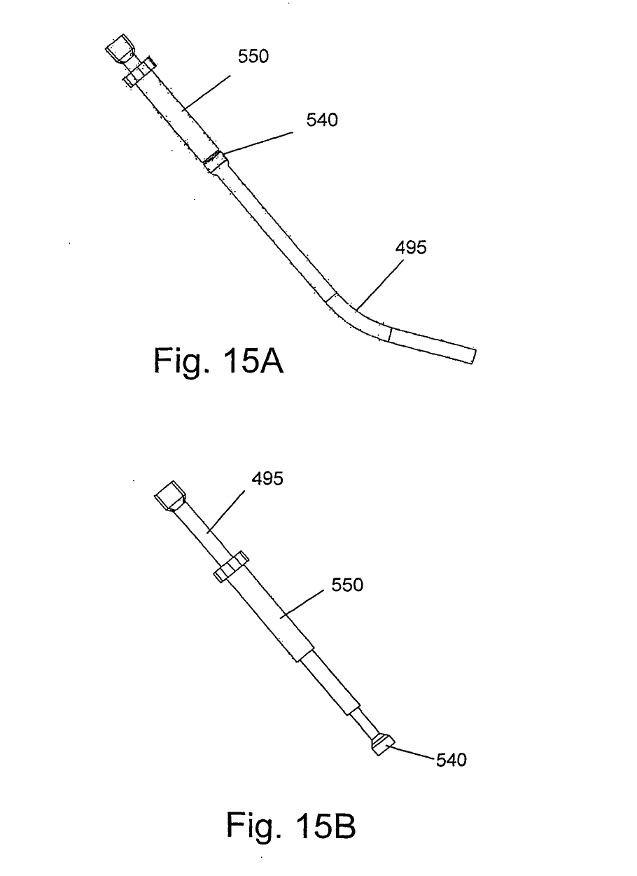

As shown in FIG. 15A, sealing plug 540 is slidingly displaceable over guide sheath 495. A guide sheath is typically introduced into an artery, as shown in FIG. 12, so that a catheter may be inserted within the sheath and guided within the artery, in close proximity of the bodily tissue of interest for examination or treatment. Sliding plug 540 is engageable with the bottom of telescoping plunger 550, e.g. by means of threading. Upon operator input, plunger 550 telescopes, as shown in FIG. 15B, and plug 540 is thereby downwardly displaced to the arterial puncture site, at which the plug applies pressure and helps to seal the puncture site.

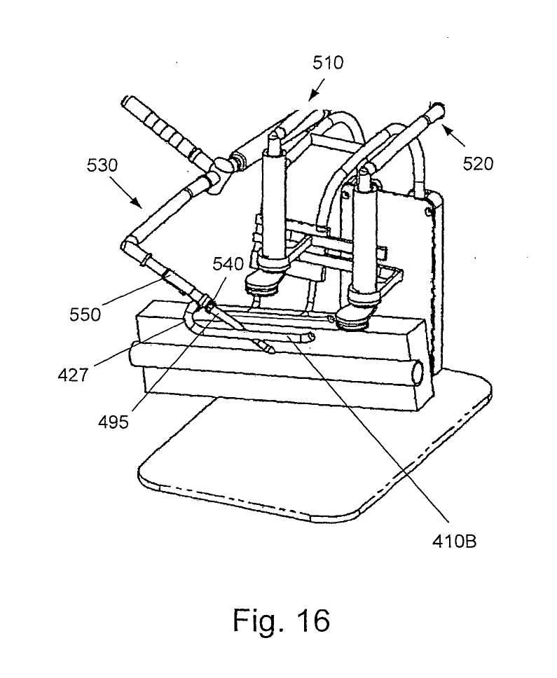

As shown in FIG. 16, a third hydraulic circuit, which is designated generally by 530, in addition to hydraulic circuits 510 and 520 for the distal and proximal plungers, respectively, is adapted to displace sliding plug 540 along sheath 495. In order to provide the reactive force needed to generate increased pressure within telescoping plunger 550, sheath 495 is supported, e.g. by clips, onto connecting bar 427, located at the distal end of tissue-confining device 410B.

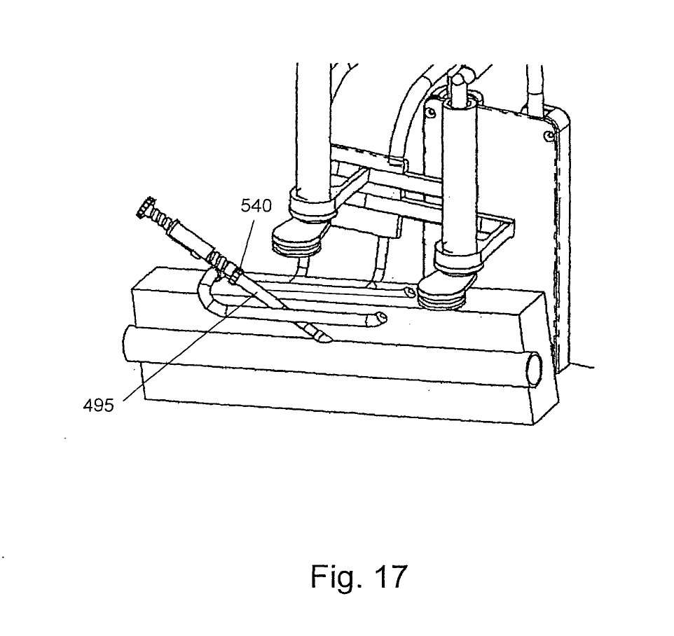

If so desired, plug 540 may be slidingly displaced along sheath 495 by mechanical means, as shown in FIG. 17.

While some embodiments of the invention have been described by way of illustration, it will be apparent that the invention can be carried into practice with many modifications, variations and adaptations, and with the use of numerous equivalents or alternative solutions that are within the scope of persons skilled in the art, without departing from the spirit of the invention or exceeding the scope of the claims.

1. An apparatus for sealing a puncture in a blood vessel comprising a tissue-confining device longitudinally extending between a proximal end and a distal end; a proximal plunger positioned adjacent to said proximal end, for adjustably applying axial pressure on the blood vessel; said tissue-confining device being connected to an adjustable artery clamp for adjustably applying pressure in an axial direction.

2. The apparatus according to claim 1, further comprising a distal plunger, said distal plunger adapted for applying axial pressure onto the blood vessel, essentially above the puncture.

3. The apparatus according to claim 1, wherein the tissue-confining device comprises two parallel, longitudinally extending bars, interconnected at or adjacent their respective proximal and/or distal ends by arcuate connecting members.

4. The apparatus according to claim 1, wherein the tissue-confining device is pivotally connected to the artery clamp.

5. The apparatus according to claim 1, wherein the tissue-confining device is releasable from the artery clamp device.

6. The apparatus according to claim 2, wherein the distal plunger is pivotally mounted on a pivotally displaceable bracket secured to the tissue-confining device.

7. The apparatus according to claim 1, wherein the tissue-confining device further comprises a seating for removably fixing thereto a transducer of an imaging device.

8. The apparatus according to claim 1, further comprising a pressure indicating means for indicating the axial pressure applied to tissue.

9. The apparatus according to claim 3, wherein one of the bars is formed with an opening for removing therethrough a guide sheath.

10. The apparatus according to claim 1, wherein the proximal plunger extends from the artery clamp device.

11. The apparatus according to claim 1, wherein the proximal plunger extends from the tissue-confining device.

12. The apparatus according to claim 2, wherein the proximal and/or distal plunger is axially displaceable.

13. The apparatus according to claim 2, wherein the proximal plunger and/or the distal plunger is longitudinally slidingly displaceable.

14. The apparatus according to claim 1, further comprising at a distal side of the tissue-confining device, a sealing plug support device for positioning and supporting a sealing plug pusher member.

15. The apparatus according to claim 14, wherein the sealing plug comprises a blood vessel engaging portion for bearing against the boundaries of the puncture, and a sealing portion slidably received over a guide sheath; said sealing portion being spontaneously sealable upon withdrawal of the guide sheath; the sealing plug being displaceable by a pusher member.

16. The sealing plug according to claim 15, wherein the pusher member is attached to the sealing plug to facilitate withdrawal thereof.

17. The sealing device according to claim 15, wherein the sealing portion is a pre-slotted resilient membrane.

18. The apparatus according to claim 3, wherein a connecting member is adapted for supporting a telescoping plunger, a sealing plug connected to the bottom of said telescoping plunger being slidable about the outer wall of a sheath insertable within a puncture site of a blood vessel.

19. The apparatus according to claim 2, further comprising means for adjusting the angular orientation, with respect to the artery clamp, of a point from which pressure is applied to the blood vessel.

20. The apparatus according to claim 1, wherein axial pressure is applied by means selected from the group of mechanical means, hydraulic means, pneumatic means and electrical means.

21. The apparatus according to claim 20, wherein axial pressure is generated by means of a fluid circuit comprising an actuator, a cylinder in which fluid is pressurized, a conduit for said fluid, and a junction by which said cylinder and said conduit are in fluid communication with one another, fluid being flowable within said fluid circuit to or from a plunger.

22. The apparatus according to claim 21, wherein the axial pressure is adjustable by means of the actuator.

23. The apparatus according to claim 21, wherein the junction is in fluid communication with a valve through which fluid is introduced to the fluid circuit and with a manometer for indicating the pressure being applied to tissue or to a blood vessel, said valve being adapted for isolating the fluid from the cylinder, the actuator and cylinder thereby being removable from the fluid circuit, said removable actuator and cylinder being adapted for actuating both the proximal and distal plungers.

24. A tissue-confining device for sealing a puncture in a blood vessel comprising two parallel, longitudinally extending bars, interconnected at or adjacent their respective proximal and/or distal ends by arcuate connecting members, said tissue-confining device suitable for compressing tissue in the vicinity of a punctured blood vessel and for being connected to an adjustable artery clamp, which is adapted for adjustably applying pressure in an axial direction onto a blood vessel.

25. The tissue-confining device according to claim 24, further comprising a distal plunger, said distal plunger adapted for applying axial pressure essentially above the puncture at the blood vessel.

26. A method for sealing a puncture in a blood vessel caused by a guide sheath, said method comprising the following steps:
a) confining the blood vessel and fixedly positioning it;
b) applying an axial force on the blood vessel upstream of the puncture, so as to cause partial occlusion thereof; and
c) withdrawing the guide sheath.

27. A method for sealing a puncture in a blood vessel caused by a guide sheath, said method comprising the following steps:
a) axially positioning over the blood vessel, at the vicinity of the puncture, an apparatus comprising a tissue-confining device, a proximal plunger positioned upstream of the puncture, said tissue-confining device connected to an adjustable artery clamp device;
b) adjustably applying an axial force at the vicinity of the puncture by said artery clamp device, to thereby confine the blood vessel;
c) adjustably applying axial pressure on the blood vessel by said proximal plunger, to reduce the blood pressure and blood flow in the blood vessel; and
d) withdrawing the guide sheath.

28. The method according to claim 27, wherein at least step c) is carried out while monitoring pressure in the blood vessel.

29. The method according to claim 26, wherein axial pressure is further applied by a distal plunger, directly over the puncture.

30. The method according to claim 26, comprising a further step wherein a puncture sealing plug is introduced and applied over the puncture.

31. The method according to claim 30, wherein the sealing plug is slid over the guide sheath, downwardly displaced and placed in pressing engagement with an arterial puncture.

32. An The method according to claim 30, wherein the sealing plug is not removed from the puncture.

33. A method for treating a pseudoaneurysm, said method comprising the following steps:
a) detecting a pseudoaneurysm and a pseudoaneurysm neck between an artery and said pseudoaneurysm;
b) confining the pseudoaneurysm or pseudoaneurysm neck;
c) fixedly positioning said pseudoaneurysm or pseudoaneurysm neck;
d) applying a controllable axial force on said artery upstream to a puncture which resulted in said pseudoaneurysm, so as to cause partial or total occlusion within said artery ; and
e) applying a controllable axial force on said pseudoaneurysm neck, thereby inducing a blood clot within said pseudoaneurysm.

34. An apparatus for controllably applying pressure onto a blood vessel, comprising:
a) a tissue-confining device for compressing tissue in the vicinity of a punctured blood vessel which longitudinally extends between a proximal end and a distal end;
b) at least one axially and longitudinally displaceable plunger for applying pressure onto said blood vessel;
c) means for positioning said at least one plunger to a first location above a skin target and directly over said blood vessel;
d) means for generating a controllable force; and
e) means for transmitting said controllable force to said at least one plunger in such a way that said at least one plunger is axially displaceable from said first location to a second location in contact with said skin target and directly above said blood vessel, and that a controllable and known pressure is applied by said at least one plunger onto said skin target, said pressure being controllable to such a degree so as to reduce blood flow velocity within said blood vessel.

35. The apparatus according to claim 34, wherein the means of generating a controllable force is selected from the group of hydraulic means, pneumatic means and electric means.
